(19) United States
(12) Patent Application Publication
(10) Pub. No.: US 2024/0374343 A1
(43) Pub. Date: Nov. 14, 2024

(54) SURGICAL MARKING DEVICE

(71) Applicant: Surgi Stamp LLC, New York, NY (US)

(72) Inventors: Sophie Bartsich, New York, NY (US); Omar Magdy Elkomy, Brooklyn, NY (US)

(73) Assignee: Surgi Stamp LLC, New York, NY (US)

(21) Appl. No.: 18/144,942

(22) Filed: May 9, 2023

(51) Int. Cl. A61B 90/00

(52) U.S. Cl. CPC A61B 90/39; A61B 2090/3933

(57) ABSTRACT

A surgical marking device includes cylindrical body having a top edge and a bottom edge, an inner ring mounted concentrically within the cylindrical body, and an ink pad on a bottom edge of the cylindrical body, the ink pad being configured to hold a quantity of ink for application to a body of a patient.

### FIELD OF THE INVENTION

The present invention relates generally to surgical procedures and, more particularly, to a device for creating a visible incision guide marking on a patient.

### BACKGROUND OF THE INVENTION

In many types of surgery, and particularly in plastic surgery, skin marking is a critical part of the procedure, as it guides the incisions for the surgery itself. Most typically, in breast surgery, the areola is marked before incision in order to produce a round and specifically sized shape. With reference to FIG. 1, most commonly, the marker consists of a metal device 10, colloquially referred to as a “cookie cutter,” with a ridged edge 12 that produces a temporary imprint on the skin. This imprint often disappears within seconds, leaving very little time to make the required incision. In cases where the patient is positioned upright and the surgeon must stand at the foot of the bed to visualize and evaluate positioning, there is no time to make this incision, and often times the imprint itself can be hard to see from that great a distance.

As a result, and for many years, surgeons have been drawing on the edge of the device with a pen to attempt to produce an ink pattern on the skin that would allow more time, precision and visibility to the mark. Invariably, the ink left from drawing on the ridged edge is barely adequate to produce the desired mark, and it is always highly irregular and often smudges.

In procedures such as abdominoplasty, the umbilicus also requires marking in order to incise around it for movement of the surrounding skin flap. While hundreds of thousands of these procedures are performed each year, there are no known devices which help to mark the umbilicus for circumscription. Conventionally, surgeons mark these areas free-hand, by “eyeballing it.” This creates extreme variability and inaccuracy. Because of the shape of the umbilicus and its position on a large irregular skin flap, it is also very easy to misread size, especially if the umbilicus itself is either large or small, with little or much depth. Furthermore, given the critical importance of symmetry in all of these procedures, being able to target axes of symmetry is crucial.

In view of the above, there is a need for a surgical marking device that allows a surgeon to reliably produce a highly visible and clearly defined marking on the skin of a patient before incision.

### SUMMARY OF THE INVENTION

It is an object of the present invention to provide a surgical marking device.

It is another object of the present invention to provide a surgical marking device for use in breast surgery.

It is another object of the present invention to provide a surgical marking device for use in abdominoplasty surgeries.

It is another object of the present invention to provide a surgical marking device that produces a highly visible and clearly defined marking on the skin of a patient. These and other objects are achieved by the present invention.

According to an embodiment of the present invention, a surgical marking device includes cylindrical body having a top edge and a bottom edge, an inner ring mounted concentrically within the cylindrical body, and an ink pad on a bottom edge of the cylindrical body, the ink pad being configured to hold a quantity of ink for application to a body of a patient.

According to another embodiment of the present invention, a surgical marking device includes a body having a top edge and a bottom edge and defining an open interior space, and an ink pad on a bottom edge of the body, the ink pad being configured to hold a quantity of ink for application to a body of a patient.

According to yet another embodiment of the present invention, a method for marking an incision location for a breast surgical procedure includes the steps of placing a device having a cylindrical body and an inner ring mounted concentrically within the cylindrical body over a nipple of a patient, receiving the nipple of the patient within the inner ring, and pressing an ink pad affixed to a bottom edge of the cylindrical body against skin of the patient to transfer ink from the ink pad to the skin of the patient.

### BRIEF DESCRIPTION OF THE DRAWINGS

The present invention will be better understood from reading the following description of non-limiting embodiments, with reference to the attached drawings, wherein below:

FIG. 1 is perspective view of a prior art marking device.

FIG. 2 a perspective view of a surgical marking device according to an embodiment of the present invention.

FIG. 3 is a bottom plan view of the surgical marking device of FIG. 2

FIG. 4 is a top plan view of the surgical marking device of FIG. 2.

FIG. 5 is a bottom plan view of a surgical marking device according to another embodiment of the present invention.

FIG. 6 is a top plan view of the surgical marking device of FIG. 5.

### DETAILED DESCRIPTION OF THE PREFERRED EMBODIMENT

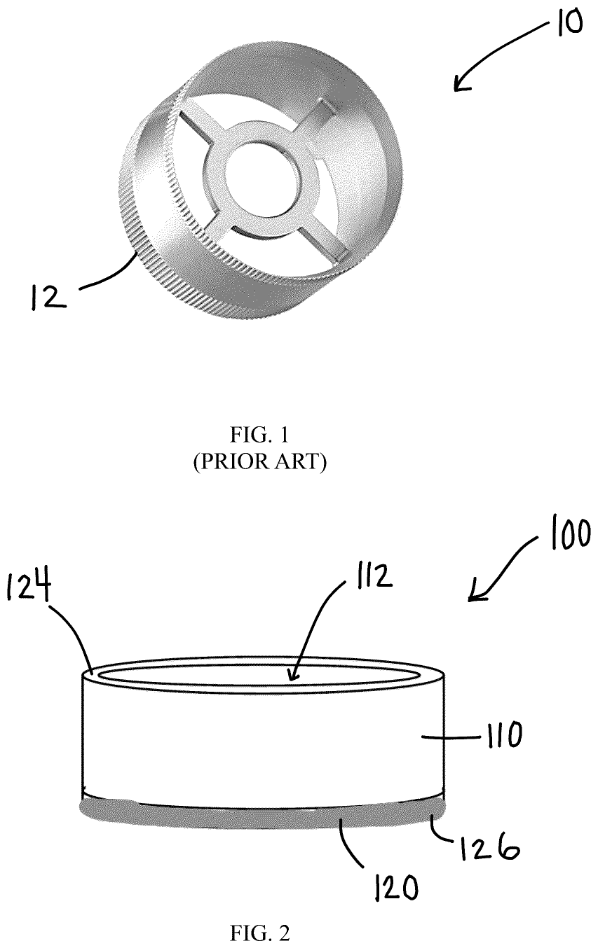

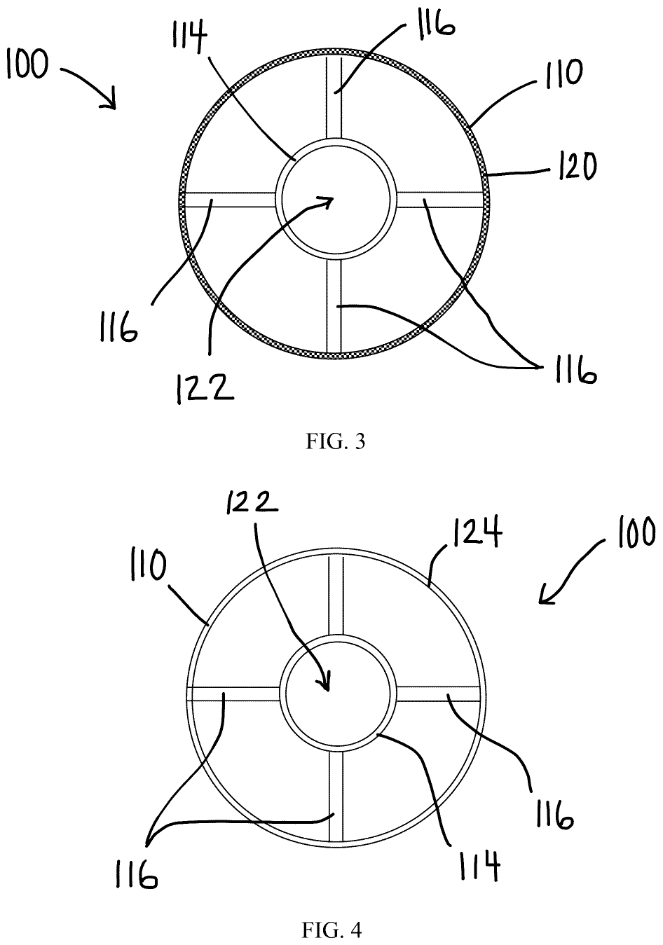

With reference to FIGS. 2-4, a surgical marking device 100 according to an embodiment of the present invention is illustrated. The marking device 100 is generally similar to the marking device 10 shown in FIG. 1, and includes a thin-walled cylindrical body 110 defining an open interior space 112, and having a top edge 124 and a bottom edge 126. Within the interior space is supported a ring 114 defining a nipple receiving area 122. In an embodiment, the ring 114 is mounted concentrically with the cylindrical body 110 by a plurality of supports 116 extending between the ring 114 and the body 110. While FIGS. 2 and 3 illustrate the use of four supports 116 spaced 90 degrees from one another, more than or fewer than four supports may be utilized without departing from the broader aspects of the invention. In an embodiment, the ring 114 is mounted halfway between the top edge and the bottom edge of the cylindrical body 110.

As best shown in FIGS. 2 and 3, the surgical marking device 100 further includes an annular, absorbent pad 120 affixed to the bottom edge of the cylindrical body 110. The pad 120 is configured to absorb and retain ink for the purpose described hereinafter. In an embodiment, the pad 120 is impregnated with a surgical ink such as, for example, gentian violet, and contains enough ink for approximately, or at least, ten stamps (although not so much ink that it would bleed or smear upon application). It is contemplated that the pad 120 may be formed from felt or a foam material, although any porous material capable of retaining a quantity ink for subsequent dispersal when pressure is applied may be utilized without departing from the broader aspects of the invention.

In an embodiment, the device 100, including the cylindrical body 110, inner ring 114 and supports 116 is formed from a transparent material such as polycarbonate, polystyrene, silicone or other material, which allows visualization of the patient's nipple and surrounding skin, and facilitates precise placement of the device 100 in concentric fashion, during use. It is also envisioned, however, that the device may be formed from or otherwise incorporate non-transparent materials such as materials such as plastics, metals and the like, without departing from the broader aspects of the invention.

In an embodiment, the cylindrical body 110 is approximately 3 centimeters in height, and has a diameter ranging from about 34 millimeters to about 42 millimeters, although other sizes are possible without departing from the broader aspects of the invention. In an embodiment, the diameter of the cylindrical body may be 34 millimeters, 36 millimeters, 38 millimeters, 40 millimeters, 42 millimeters, or 44 millimeters. Regardless of the diameter of the cylindrical body 110, the inner ring has an inside diameter of approximately 1.5 centimeters. In an embodiment where the cylindrical body 110 is 3 centimeters in height, the inner ring 120 is located approximately 0.5 to 1.5 centimeters and, more preferably, 1.5 centimeters from the bottom edge of the cylindrical body 110.

In use, the device 100 is used as a mammary marker during breast surgeries, and is placed on a patient's breast so that the inner ring 114 is placed over the base of the patient's nipple. As indicated above, the transparent nature of the device 100 allows for precise placement and visualization of the patient's nipple during use. This is in contrast to existing devices which are non-transparent, which can impede visualization and property placement. After the inner ring 114 is received around the patient's nipple, gentle pressure may be applied to press the device 100 against the patient's skin to transfer ink from the pad 120 to the patient's skin, leaving a defined and easily visible circular mark on the patient. After marking, a surgeon move to the foot of the bed and sit the patient up in order to evaluate placement. In contrast to existing devices and methods, the ink circle left by the device 100 is well defined and clearly visible from the foot of the bed. It also does not dissipate from the time it is placed, to the time the incision is made. In stark contrast, existing devices which rely on an imprint or abrasion of the skin can dissipate quickly, making the mark difficult to see, or otherwise damages or injures the skin of the patient. The device 100 is intended to be discarded after use.

In embodiments, where the pad 120 is impregnated with ink, no separate ink reservoir is required. It is contemplated, however, that the pad 120 may be sold and delivered dry, and a surgeon may press or dip the pad 120 into a separate ink reservoir to coat the ink pad 120 before use. While FIGS. 1-4, illustrate a single absorbent ink pad 120 on the bottom edge of the device, the present invention is not intended to be so limited in this regard. In particular, it is contemplated that the top edge of the device 100 may include a second ink pad (not shown) or may include a plurality of ridges which may be used to create a mark or imprint on a patient's skin. In embodiments where the top edge includes ridges, the top edge can be used to create a faint imprint when determining initial placement, and then the device 100 may be flipped over and the ink pad 120 utilized to create a more lasting mark.

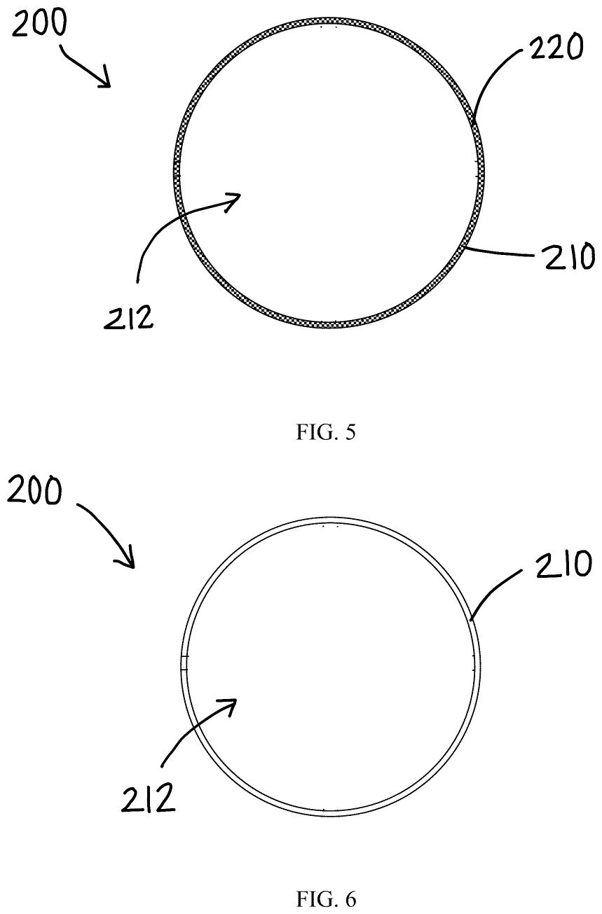

Turning now to FIGS. 5 and 6, a surgical marking device 200 according to another embodiment of the present invention is illustrated. The marking device 200 is similar to marking device 100 and includes a thin-walled cylindrical body 210 defining an open interior space 212. In an embodiment, the body 210 has a diameter ranging from about 1.5 centimeters to about 3 centimeters. In an embodiment, the diameter of the cylindrical body may be 1.5 centimeters, 2 centimeters, 2.5 centimeters, or 3 centimeters. The height of the cylindrical body 210 may be approximately 3 centimeters. Notably, the device does not contain an inner ring or supports. Similar to device 100, device 200 includes an annular, absorbent pad 220 affixed to the bottom edge of the cylindrical body 210. The pad 220 is configured to absorb and retain ink. In an embodiment, the pad 220 is impregnated with a surgical ink such as, for example, gentian violet, and contains enough ink for approximately, or at least, ten stamps (although not so much ink that it would bleed or smear upon application). It is contemplated that the pad 220 may be formed from felt or a foam material, although any porous material capable of retaining a quantity ink for subsequent dispersal when pressure is applied may be utilized without departing from the broader aspects of the invention.

Like device 100, device 200 may be formed from a transparent material of the type disclosed above. In an embodiment, the device 200 is configured as an umbilical marker for use in creating a mark for incision around the umbilicus for use during abdominoplasty surgeries. While FIGS. 5 and 6 illustrate the device 200 as being cylindrical in shape, it is contemplated that the device may be oval, triangle or other shapes.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of this disclosure.

What is claimed is:

1. A surgical marking device, comprising:
a cylindrical body having a top edge and a bottom edge;
an inner ring mounted concentrically within the cylindrical body; and
an ink pad on a bottom edge of the cylindrical body, the ink pad being configured to hold a quantity of ink for application to a body of a patient.

2. The surgical marking device of claim 1, wherein:
the ink pad is impregnated with ink.

3. The surgical marking device of claim 2, wherein:
the ink is gentian violet.

4. The surgical marking device of claim 1, wherein:
the ink pad is formed from an absorbent material.

5. The surgical marking device of claim 1, wherein:
the cylindrical body has a height of approximately 3 centimeters and the inner ring is mounted at an approximate midpoint between the top edge and the bottom edge.

6. The surgical marking device of claim 5, further comprising:
a plurality of supports extending between the inner ring and the cylindrical body for supporting the inner ring within an open interior space defined by the cylindrical body.

7. The surgical marking device of claim 1, wherein:
the cylindrical body is formed from a transparent material.

8. The surgical marking device of claim 7, wherein;
the cylindrical body is formed from polycarbonate, polystyrene or silicone.

9. The surgical marking device of claim 6, wherein:
the cylindrical body, the inner ring and the plurality of supports are formed from a transparent material.

10. The surgical marking device of claim 1, wherein:
the inner ring has an inside diameter of 1.5 centimeters.

11. The surgical marking device of claim 10, wherein:
the cylindrical body has a diameter between about 34 millimeters and about 42 millimeters.

12. The surgical marking device of claim 1, wherein:
the top edge includes a plurality of ridges.

13. A surgical marking device, comprising:
a body having a top edge and a bottom edge and defining an open interior space; and
an ink pad on a bottom edge of the body, the ink pad being configured to hold a quantity of ink for application to a body of a patient.

14. The surgical marking device of claim 13, wherein:
the ink pad is impregnated with ink.

15. The surgical marking device of claim 14, wherein:
the ink is gentian violet.

16. The surgical marking device of claim 13, wherein:
the ink pad is formed from an absorbent material.

17. The surgical marking device of claim 13, wherein:
the body has a circular cross section.

18. The surgical marking device of claim 13, wherein:
the body has one or a triangular cross section or an oval cross section.

19. A method for marking an incision location for a breast surgery, the method comprising the steps of:
placing a device having a cylindrical body and an inner ring mounted concentrically within the cylindrical body over a nipple of a patient;
receiving the nipple of the patient within the inner ring; and
pressing an ink pad affixed to a bottom edge of the cylindrical body against skin of the patient to transfer ink from the ink pad to the skin of the patient.

20. The method according to claim 19, wherein:
the cylindrical body is formed from a transparent material.
